(19) United States
(12) Patent Application Publication
(10) Pub. No.: US 2006/0197664 A1
(43) Pub. Date: Sep. 7, 2006

(54) Method, system and apparatus for a time stamped visual motion sensor

(75) Inventors: Guangbin Zhang, Sunnyvale, CA (US); Jin Liu, Frisco, TX (US)

Correspondence Address: CHALKER FLORES, LLP, 2711 LBJ FRWY, Suite 1036, DALLAS, TX, 75234, US

(73) Assignee: Board Of Regents, The University Of Texas System, Austin, TX (US)

(21) Appl. No.: 11/335,235

(22) Filed: Jan. 18, 2006

Related U.S. Application Data
(60) Provisional application No. 60/644,822, filed on Jan. 18, 2005.

(51) Int. Cl. G08B 13/18; C12Q 1/68; G06M 7/00; G08B 21/00; G06K 9/00; H04N 7/18

(52) U.S. Cl. 340/555; 340/686.1; 250/200; 250/221; 348/155; 382/103

(57) ABSTRACT

The present invention provides a method, system and apparatus for a time stamped visual motion sensor that provides a compact pixel size, higher speed motion detection and accuracy in velocity computation, high resolution, low power integration and reduces the data transfer and computation load of the following digital processor. The present invention provides a visual motion sensor cell that includes a photosensor, an edge detector connected to the photosensor and a time stamp component connected to the edge detector. The edge detector receives inputs from the photosensor and generates a pulse when a moving edge is detected. The time stamp component tracks a time signal and samples a time voltage when the moving edge is detected. The sampled time voltage can be stored until the sampled time voltage is read. In addition, the edge detector can be connected to one or more neighboring photosensors to improve sensitivity and robustness.

### FIELD OF THE INVENTION

The present invention relates generally to the field of visual motion detection, and more particularly to a method, system and apparatus for a time stamped visual motion sensor.

### BACKGROUND OF THE INVENTION

Visual motion information is very useful in many applications such as high speed motion analysis, moving object tracking, automatic navigation control for vehicles and aircrafts, intelligent robot motion control, and real-time motion estimation for MPEG video compression. Traditional solutions use a digital camera plus digital processor or computer system. The digital camera captures the video frame by frame, transfers all the frame data to digital processor or computer and calculates the motion information using image processing algorithms, such as block matching. However, both the motion computation load and the data transfer load between the camera and the processor for large scale 2-D arrays are very high.

For example, a MPEG4 CIF resolution 352×288 video needs to be sampled at a rate of 1,000 frames per second (fps) to detect motion in 1/1,000 second time resolution. If the video frame is an 8-bit monochrome image, the data transfer rate between the camera and the computer should be larger than 8×10⁸ bps. To extract basic motion information for each pixel, the computer should at least compare each pixel with its four nearest neighbor pixels in the previous frame. This leads to a computational load as high as 4×10⁸×T, where T is the time required for comparing one pair of pixels. Obviously, this is such a heavy load that there is very small computational resource left for the computer to perform other tasks required by the system application. Note that some scientific or industrial ultra-high speed motion analysis requires 10,000 frames per second or more. For more reliable results, an imaging processing algorithm such as block-matching may be used, which leads to even higher computational load. As a result, the required computational resources may exceed the power of most computers. Moreover, the power consumption associated with the load is often prohibitive, especially for battery powered portable devices.

To reduce the load and the power consumption, smart visual motion sensors with in-pixel processing circuits have been developed during the past twenty years. These systems allow pixel level parallel processing and only transfer out the extracted information. The corresponding data transfer load and computation load can potentially be reduced to several hundreds of times lower than traditional digital camera systems. However, there are still more issues to be resolved. The first is in the calculation of the motion speed. When the velocity is calculated based on RC constants of each pixel, the mismatch between the different pixels and the random noise make the calculated velocity inaccurate. The second issue is the limited measurable speed range. The intro-scene dynamic range for speed measurement is limited by the voltage swing. With linear representation, normally only two decades can be achieved. Log scale representation may be used to obtain wider dynamic range, but the precision will be largely dropped as a tradeoff. The third issue is in the readout of the motion information. When sending out the speed vectors in each frame, the motion detectors can lose the information of the exact time point when the motion occurs within one frame. This limits the performance of the motion sensor.

With respect to the first issue (motion speed calculation), there are three major categories of algorithms for velocity calculation in visual motion sensors: intensity gradient based, correlation based, and feature based. The simplest format of the feature based algorithm is the edge based motion detection, which basically uses the image edge as the feature of the object. FIG. 1 illustrates the basic “time of travel” algorithm for calculating the velocity using edge based motion sensors. The motion is detected by tracking the edge disappearing at one place (e.g., pixel one) and reappearing at another place (e.g., pixels two and three). By measuring the distance between the two places (e.g., distance, d, between pixel one and two) and the travel time (e.g., t₂−t₁), the object speed can be calculated (e.g., v=d/(t₂−t_{1 )=d/(t}₃−t₂). The shape and location of the moving object can also be identified when integrating the pixels into a high resolution 2D array.

The first chip implementation was reported in 1986. Since then there are many designs reported in 1D or 2D format, based on the gradient, correlation, or feature based algorithms, respectively. Some of them introduced biological inspired model structures to enhance the performance. Researchers have successfully used them in object tracking, velocity measurement and aiding autonomous driving of miniature vehicles and aircrafts. However, the additional processing circuits for pixel level motion computation normally result in large pixel size and high pixel power consumption, which largely limits the use of this kind of sensors. Furthermore, the accuracy of measured motion velocity is also not good enough for some applications.

There are two choices in implementing “time of travel” algorithm, i.e. to calculate the velocity within each pixel or to transfer the time points out of the array and calculate the velocity by digital processor. The facilitate-and-sample (FS) algorithm is used to calculate the velocity within each pixel. The basic pixel structure 200 for the FS algorithm is illustrated in FIG. 2A and includes photo sensors 200, an edge detector 202 and a speed calculation unit 206. Pixel structure 200 uses an in-pixel charging or discharging process to convert the travel time to a voltage and then output its value to the world. As shown in FIGS. 2B and 2C, the edge detector 204a generates a short pulse 208a when detecting substantial light density change at this pixel 202a. The edge detector 204a also generates a slow pulse 210a that starts at a high voltage and then discharges slowly. The slow pulse 208a is used as the facilitate pulse and the short pulse 210a is used as the sampling pulse. When another moving edge is detected at its neighbor 204b, the sample-and-hold circuit 206b samples the voltage of the slow pulse 208a and uses it for output 212b. The output voltage 212a or 212b represents the velocity of the object toward left or right. Some variation or additional circuits may be needed to suppress the null direction or make it more robust.

Although the FS architecture 200 can detect speed at each pixel, there are several major problems that prevent it to be used in real industrial or commercial products. First, due to the serious mismatch and nonlinearity of the CMOS process, the detected speed is very inaccurate. Second, the time constant of the charge or discharge process in each pixel is fixed during the testing, so the detectable dynamic range for the speed is very limited, i.e. it is not able to detect fast motion and slow motion at the same time. In addition, the transient time for the obtained speed is ambiguous, the exact time when the moving happened within one frame period is not known. This loss of information may be critical for some real time applications. Other implementations of edge based velocity sensor are normally similar to this method, using the in-pixel charging/discharging for time-to-voltage conversion.

To avoid the inaccuracy introduced by in-pixel time-to-voltage conversion, alternative methods have been developed. The Facilitate-Trigger-Inhibition (FTI) algorithm is used to directly output a pulse whose width is the travel time between neighbor pixels. FIG. 3A illustrates the basic pixel structure 300 for FTI algorithm, which includes photosensors 302, an edge detector 304 and a time to square wave converter 306. The accuracy of the FTI pixel structure 300 is better and less dependent on the pixel mismatches. However, to obtain the velocity of the moving edges, the width of the output pulse needs to be measured. For a high integrated 2D array, it is not possible to measure the width of the pulse directly for each pixel. The pixel array outputs need to be read out frame by frame before measuring. Thus, the time resolution is still limited by the readout frame rate, as is the accuracy of speed measurement.

More specifically, the FTI pixel structure 300 uses the signals from three adjacent edge detectors to calculate speed. As shown in FIGS. 3B and 3C, a pulse Iᵢ₋₁ from edge detector 304(i−1) at photosensor 302(i−1) facilitates the circuit for rightward motion 306(right). When the edge arrives at photosensor 302(i), a pulse Iᵢ from the edge detector 304(i) triggers the rightward motion detector 306(right) and Vᵣ jumps to logic high. Vᵣ will continue to keep high until the edge arrives the photosensor 302(i+1), when an inhibitory pulse Iᵢ₊1 is generated by edge detector 304(i+1). Hence, the pulse width of Vᵣ is inversely proportional to the velocity of the moving edge. Although the pulse width for the detected Vᵣ is accurate and much less dependent on the circuit mismatches, there are still some problems which limit the use of this kind of motion sensor. Firstly, to measure the velocity of the moving edges, the width of the Vₚᵤₗₛₑ needs to be measured. For a high integrated two dimensional array, the width of Vₚᵤₗₛₑ cannot be measured directly for each pixel. The pixels can only be read out frame by frame and then measured. That is, the accuracy of the speed measurement is still limited by the read out frame rate. Secondly, since it is necessary to read out the sensor outputs at a very high frame rate to improve the accuracy of the speed measurement, it will occupy a big amount of time of the following computer or digital processor. This also requires high bit rate data communication and high power consumption.

Another solution is to use an event driven method for readout. The basic pixel structure 400 is illustrated in FIG. 4 and includes photosensors 402, an edge detector 404 and an event driven IO circuit 406. Each pixel generates an output signal to request for the common output data lines when moving edge occurs. A chip level process unit record the event time together with the pixel position. Arbiter tree or WTA (Winner-take-all) architecture 408 may be used to resolve event conflicts with other pixels 410, i.e. edge occurrences at the same time point. This architecture has the advantages of more accurate transient time recording since it does not depend on in-pixel RC constants. It also has low power consumption because most part of the circuit works in static mode when that part of the scene is not moving. However, a major problem of this method is event confliction, which is serious when the array size is large. It is very common that there is a large object or many objects moving in the scene simultaneously. In that case, a big amount of edges can occur within a very short period of time, which could exceed the maximum bandwidth of the output interface. Even if the events are still been recorded after conflict-solving, the recorded event occurring time is not accurate because of the extra delay caused by the confliction.

There is, therefore, a need for a method, system and apparatus for a time stamped visual motion sensor that provides a compact pixel size, higher speed motion detection and accuracy in velocity computation, high resolution, low power integration and reduces the data transfer and computation load of the following digital processor.

### SUMMARY OF THE INVENTION

The present invention provides a method, system and apparatus for a time stamped visual motion sensor that provides a compact pixel size, higher speed motion detection and accuracy in velocity computation, high resolution, low power integration and reduces the data transfer and computation load of the following digital processor. More specifically, the present invention provides a new pixel structure based on a time stamped architecture for high-speed motion detection that solves many of the problems found in prior art devices. The relatively simple structure of the present invention, as compared to prior art structures, provides a compact pixel size results in a high resolution, low power integration. Moreover, the present invention does not use an in-pixel velocity calculation unit or an event-driven signaling circuit. Instead, the present invention uses an in-pixel time stamp component to record the motion transient time. Each pixel records the transient time of the motion edges asynchronously and then the information are read out frame by frame for post processing.

Measurement results show that the visual motion sensor using the time stamped architecture can detect motion information at 100 times higher time resolution than the frame rate. This enables much higher speed motion detection and greatly reduces the data transfer and computation load of the following digital processor. Moreover, the present invention can detect a wider range of motion speed by combining the timestamps in many consecutive frames together. As a result, the present invention can detect very fast and very slow movements (less than one pixel per sample period) at the same time without adjusting any device parameters or control signals. In addition, this structure is less sensitive to pixel mismatches and does not have the readout bottleneck problems found in FTI and event-driven signaling structures. As a result, the present invention provides higher accuracy in velocity computation with smaller pixel size and lower power consumption

More specifically, the present invention provides a visual motion sensor cell that includes a photosensor, an edge detector connected to the photosensor and a time stamp component connected to the edge detector. The edge detector receives inputs from the photosensor and generates a pulse when a moving edge is detected. The time stamp component tracks a time signal and samples a time voltage when the moving edge is detected. The sampled time voltage can be stored until it is read. In addition, the edge detector can be connected to one or more neighboring photosensors to optimize its sensitivity and robusticity.

The time stamp component may include a capacitor, a first, second, third and fourth switches, and a first and second D-flip-flop. The first switch is connected in series between a time input and the parallel connected capacitor. The second switch is connected in series between the parallel connected capacitor and the third switch. The third switch is controlled by a read signal and connected in series to a source follower, which is connected in series to an output node. The fourth switch is controlled by the read signal and connected in series between the output terminal of the second D-flip-flop and an odd frame signal node. The first D-flip-flop has a clear terminal that receives a reset signal, a clock terminal connected to the edge detector, a data terminal connected to a voltage source, a first output terminal that supplies a first output signal to control the first switch and a second output terminal that supplies an inverted first output signal to control the second switch. The second D-flip-flop has a clock terminal that receives the first control signal from the first D-flip-flop, a data terminal that receives an odd-even frame signal and an output terminal that supplies an inverted second output signal. Note that the second D-flip-flop can be replaced by storing the digital value onto with a transistor gate capacitor, which further reduces the layout area.

The motion sensor cells of the present invention can also be integrated into a 2D array of pixel groups. Each pixel group includes a first pixel that is sensitive to a bright-to-dark edge in a X direction, a second pixel that is sensitive to the bright-to-dark edge in a Y direction, a third pixel that is sensitive to a dark-to-bright edge in the X direction and a fourth pixel that is sensitive to the dark-to-bright edge in the Y direction. Identical temporal edge detectors can be chosen all cells too. The temporal edge detector detects the sudden changes in a single pixel itself. The major advantage of using temporal edge detector is the smaller layout size. However, this embodiment is not suitable for environments with strong flashing light(s).

In addition, the present invention provides a visual motion sensor chip that includes an array of visual motion cells, an X-axis and Y-axis scanner, a multiplexer, a synchronization signal generation logic and output buffer, and an input buffer and synchronization logic circuits. Each visual motion cell includes a photosensor, an edge detector connected to the photosensor, and a time stamp component connected to the edge detector and provides an output signal. The X-axis scanner is connected to the array of visual motion cells. The Y-axis scanner connected to the array of visual motion cells. The multiplexer is connected to the array of visual motion cells and that provides a time output, an image output and an odd frame output. The synchronization signal generation logic and output buffer provides a vertical synchronization signal, a horizontal synchronization signal and a pixel clock signal, and is connected to the X-axis scanner and the Y-axis scanner. The input buffer and synchronization logic receives an odd-even frame signal, a time signal and a clock signal, and is connected to the X-axis scanner, the array of visual motion cells and the multiplexer. The visual motion sensor chip can be integrated into a device used for video compression, robotics, vehicle motion control or high speed motion analysis.

Moreover, the present invention provides a method of detecting visible motion by receiving an image signal from a photosensor, tracking a time signal, determining whether a moving edge is detected in the image signal and sampling a time voltage from the time signal when the moving edge is detected. The method may also include storing the sampled time voltage and outputting the sampled time voltage when a read signal is received. Likewise, the method may include estimating a motion of a visible object by comparing the sampled time voltages from an array of photosensors.

For example, a demo 32×32 visual motion sensor based on the present invention has been fabricated. It has-a pixel size of 70 μm×70 μm in a standard 0.35 μm CMOS process. Such a device can measure up to 6000 degree/s with a focal length f=10 mm and has less than 5% rms variation for middle range velocity measurement (300 to 3000 degree/s) and less than 10% rms variation for high velocity (3000 to 6000 degree/s) and low velocity (1 to 300 degree/s) measurement. The device has a power consumption of less than 40 μW/pixel using a single power supply. This structure is good for scaling down with new fabrication processes to implement large scale 2D arrays with low power consumption. Other characteristics of the device include a fill factor greater than or equal to 32%, a frame readout rate greater than or equal to 100 fps, a peak time resolution less than or equal to 77 μs at 100 fps with 3000 degrees/s input, and a dynamic range for luminance of 400 to 5000 Lux at larger than 50% pixel response rate at 50% input contrast with a lens F-number 1.4.

Other features and advantages of the present invention will be apparent to those of ordinary skill in the art upon reference to the following detailed description taken in conjunction with the accompanying drawings.

### BRIEF DESCRIPTION OF THE DRAWINGS

The above and further advantages of the invention may be better understood by referring to the following description in conjunction with the accompanying drawings, in which:

FIG. 1 is a diagram illustrating the basic algorithm for velocity calculation in edge based visual motion sensors;

FIGS. 2A, 2B and 2C illustrate a motion sensor pixel structure and its operation based on a FS algorithm in accordance with the prior art;

FIGS. 3A, 3B and 3C illustrate a motion sensor pixel structure and its operation based on a FTI algorithm in accordance with the prior art;

FIG. 4 illustrates a motion sensor pixel structure based on an event-driven algorithm in accordance with the prior art;

FIG. 5 illustrates a motion sensor pixel structure based on a time stamped algorithm in accordance with one embodiment of the present invention;

FIG. 6 is a flow chart of a method to detect visible motion in accordance with one embodiment of the present invention;

FIG. 7 illustrates a multi-point linear fit for velocity calculations in accordance with the present invention;

FIG. 8 is a schematic diagram of a time stamp component in accordance with one embodiment of the present invention;

FIG. 9 is a schematic diagram of a time stamp component showing more details at the transistor level in accordance with one embodiment of the present invention;

FIG. 10 is a simulated waveform of the time stamp component in accordance with one embodiment of the present invention;

FIG. 11 shows the structure for the two dimensional motion sensor cell in accordance with one example of the present invention;

FIG. 12 shows the chip layout using the structure of FIG. 11 in accordance with one example of the present invention;

FIG. 13 shows the measured “out” and “odd” frame signals of the time stamped motion sensor pixel of FIG. 11 in accordance with one example of the present invention;

FIG. 14 shows a graph of the measured time stamp output versus moving edge occurring time of the time stamped motion sensor pixel of FIG. 11 in accordance with one example of the present invention;

FIG. 15 shows a system architecture used to test the 2-D sensor array of FIG. 11 in accordance with one example of the present invention;

FIG. 16 shows ten frames of sampled time stamps using the system architecture of FIG. 15 in accordance with one example of the present invention;

FIG. 17 illustrates a compact spatial based edge detector in accordance with one embodiment of the present invention;

FIG. 18 is a graph illustrating a comparison of contrast sensitivity distribution for a compact spatial based edge detector in accordance with one embodiment of the present invention and a prior art edge detector;

FIGS. 19A and 19B illustrate the time stamp recording process using square and narrow par shape photosensors in accordance with one embodiment of the present invention;

FIG. 20 illustrates a double edge problem observed in some motion sensor chips;

FIG. 21 is a layout pattern of a spatial edge based time stamped motion sensor in accordance with one embodiment of the present invention;

FIG. 22 is a chip block diagram and readout structure in accordance with one embodiment of the present invention;

FIG. 23 depicts a measured pixel response rate of the spatial edge detector in 2D array (with 50% contrast input) in accordance with one embodiment of the present invention;

FIG. 24 depicts a measured velocity in horizontal and vertical directions in accordance with one embodiment of the present invention;

FIG. 25 depicts an equivalent time resolution based on measured velocity accuracy in accordance with one embodiment of the present invention;

FIG. 26A depicts a measured 2-D optical flow of a moving hand in accordance with one embodiment of the present invention;

FIG. 26B depicts a measured 2-D optical flow of a fast rotating fan in accordance with one embodiment of the present invention;

FIG. 27 shows a photo of a chip in accordance with one embodiment of the present invention;

FIG. 28 shows a schematic of a nano-power edge detector in accordance with one embodiment of the present invention;

FIG. 29 shows a schematic of a nano-power time stamp component in accordance with one embodiment of the present invention;

FIG. 30 shows a chip photo of an ultra-low power embodiment of the present invention;

FIG. 31 depicts a measured time stamp data from the sensor in accordance with one embodiment of the present invention;

FIG. 32A depicts a comparison of data transfer load in accordance with one embodiment of the present invention;

FIG. 32B depicts a comparison of minimum computational speed required in accordance with one embodiment of the present invention;

FIG. 33 depicts the MPEG motion estimation searching area using full search algorithm in accordance with one embodiment of the present invention;

FIG. 34 depicts the MPEG motion estimation searching area using the timestamp motion sensor of the present invention; and

FIG. 35 shows a comparison of the motion block searching area using standard full search algorithm and using time stamped visual motion sensor of the present invention.

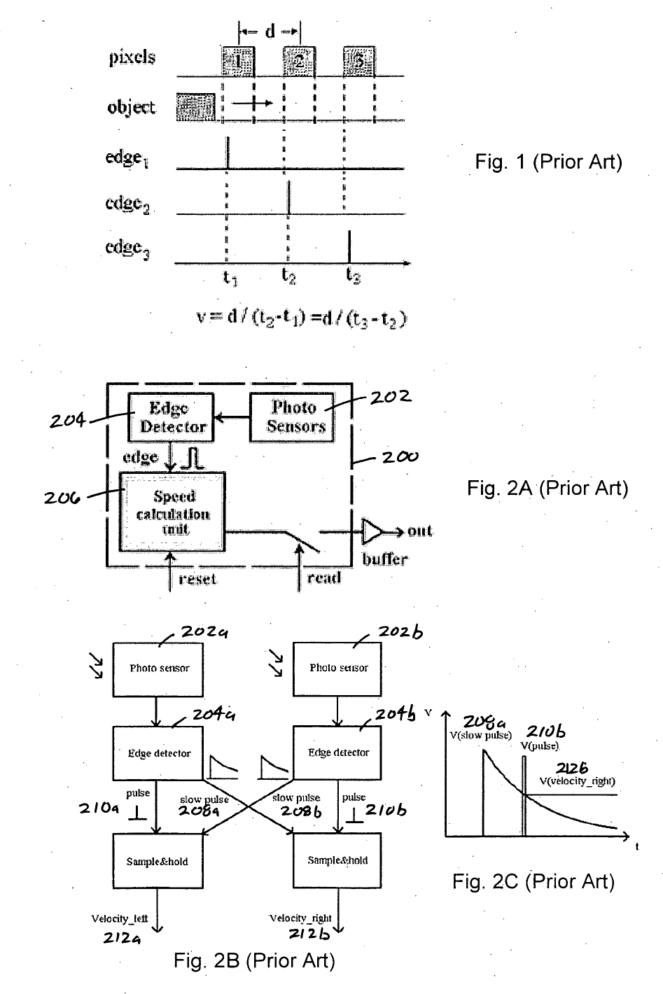

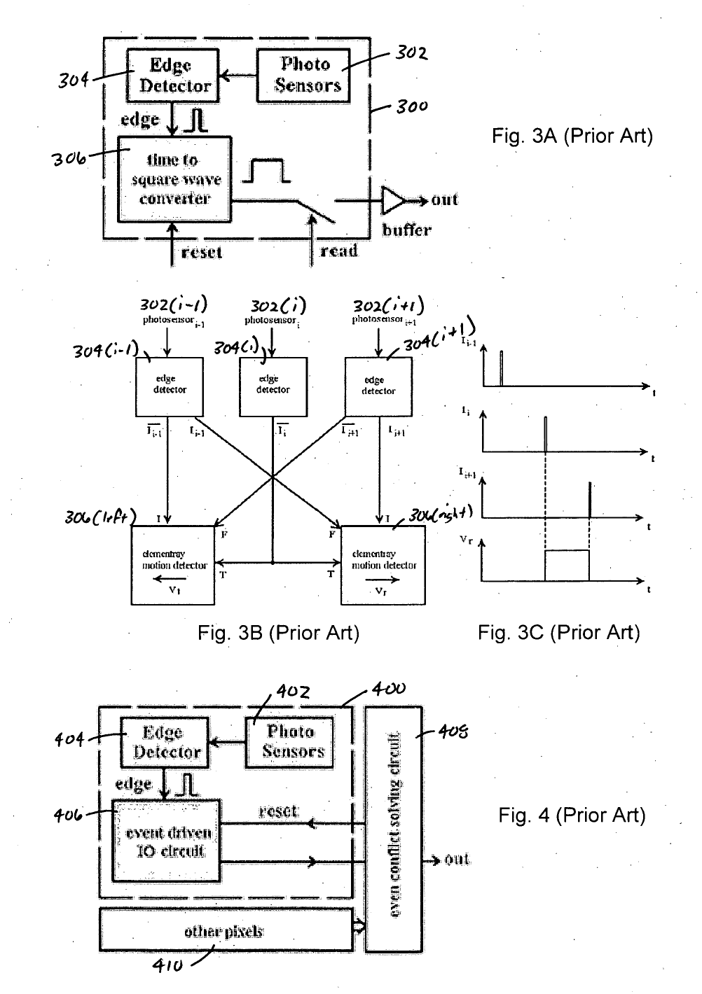

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

The present invention provides a method, system and apparatus for a time stamped visual motion sensor that provides a compact pixel size, higher speed motion detection and accuracy in velocity computation, high resolution, low power integration and reduces the data transfer and computation load of the following digital processor. More specifically, the present invention provides a new pixel structure based on a time stamped architecture for high-speed motion detection that solves many of the problems found in prior art devices. The relatively simple structure of the present invention, as compared to prior art structures, provides a compact pixel size results in a high resolution, low power integration. Moreover, the present invention does not use an in-pixel velocity calculation unit or an event-driven signaling circuit. Instead, the present invention uses an in-pixel time stamp component to record the motion transient time. Each pixel records the transient time of the motion edges asynchronously and then the information are read out frame by frame for post processing.

Measurement results show that the visual motion sensor using the time stamped architecture can detect motion information at 100 times higher time resolution than the frame rate. This enables much higher speed motion detection and greatly reduces the data transfer and computation load of the following digital processor. Moreover, the present invention can detect a wider range of motion speed by combining the timestamps in many consecutive frames together to produce a wide dynamic range. As a result, the present invention can detect very fast and very slow movements (less than one pixel per sample period) at the same time without adjusting any device parameters or control signals. In addition, this structure is less sensitive to pixel mismatches and does not have the readout bottleneck problems found in FTI and event-driven signaling structures. As a result, the present invention provides higher accuracy in velocity computation (e.g., <5% precision) with smaller pixel size and lower power consumption

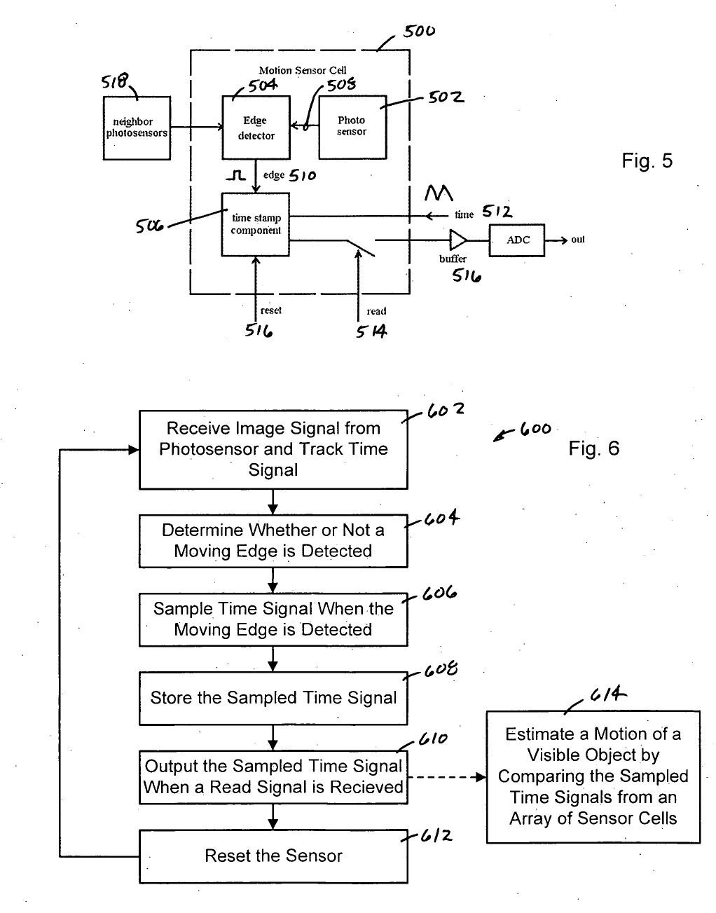

Now referring to FIG. 5, a motion sensor pixel or cell structure 500 based on a time stamped algorithm in accordance with one embodiment of the present invention is shown. The visual motion sensor cell 500 includes a photosensor 502, an edge detector 504 connected to the photosensor 502 and a time stamp component 506 connected to the edge detector 504. The photosensor 502 can be one or more phototransistors or photodiodes. The edge detector 504 receives inputs 508 from the photosensor 502 and generates a transient voltage pulse 510 when a moving edge is detected. The time stamp component 506 tracks a global time signal 512 and samples a time voltage when the moving edge is detected. The sampled time voltage can be stored until a read signal 514 is received and the sampled time voltage is provided to the output 516. The cell 500 is reset when a reset signal 516 is received. In addition, the edge detector 504 can be connected to one or more neighboring photosensors 518 to improve the sensitivity and the robustness of the edge detector by spatial and temporal adaptations.

Referring now to FIG. 6, a method 600 of detecting visible motion in accordance with one embodiment of the present invention is shown. An image signal is received from a photosensor and a time signal is tracked in block 602. Whether or not a moving edge is detected in the image signal is determined in block 604. A time voltage is sampled from the time signal when the moving edge is detected in block 606. The sampled time voltage is stored in block 608 and the sampled time voltage is provided to an output when a read signal is received in block 610. After the sampled time voltage has been read, the sensor is reset in block 612. The sampled time voltages from an array of photosensors can be compared to estimate a motion of a visible object in block 614.

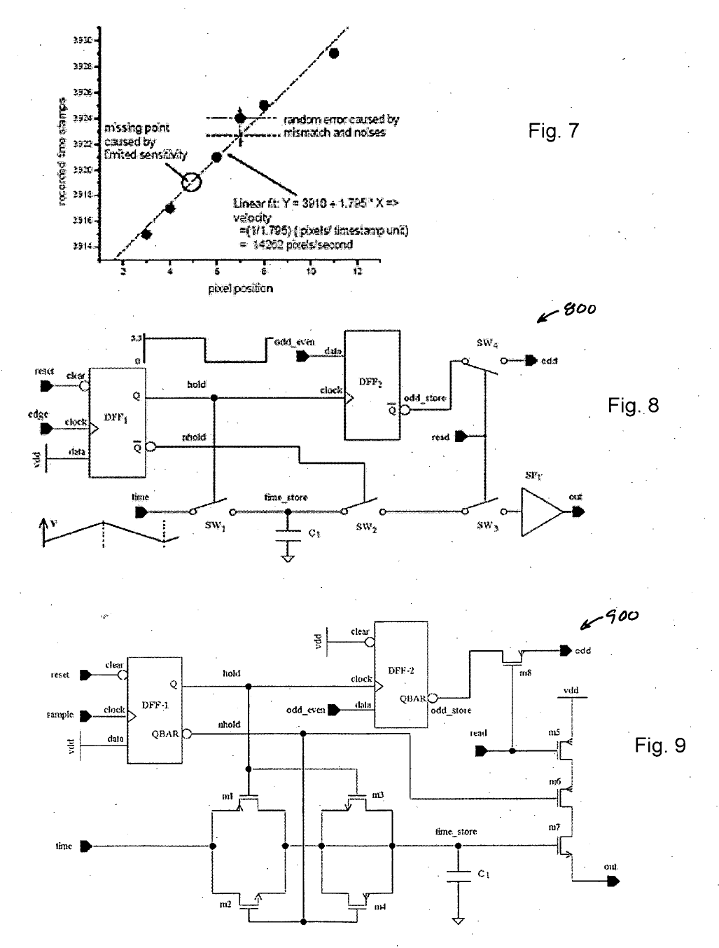

The motion velocity can be calculated by digital processor based on the obtained time stamps from the sensor. The basic formula is V=d/(t₁−t₂), where Vis the velocity, d is the distance between two pixels, and the t₁ and t₂ are two recorded time stamps. Unlike previous edge based visual motion sensors which normally use two points to calculate speed, the time stamped vision sensor of the present invention can use a multi-point linear fit to calculate speed, which is less sensitive to mismatches, noises, and missing data points. The results are more reliable and accurate. As shown in FIG. 7, several consecutive time stamp points are recorded, which indicate something moving through these points. Using a simple first order linear fit, the slope of these points, which is proportional to the moving speed, can be found.

Pixel level test results verified that the present invention can detect fast motion in more than 100 times higher resolution than the frame rate, without increasing the data throughput. Two other major advantages of the time stamped structure are the compact pixel size and low pixel power consumption, which are essential in large scale implementation and portable devices. Using the same pixel from this design, a MPEG4 CIF 352×288 format sensor will cost 24.6 mm×20.2 mm area. Previous CMOS visual motion sensors normally have in-pixel RC components, active filters or amplifiers, which are hard to scale down. Unlike such prior art sensors, the time stamped vision sensor pixel mainly contains minimum size transistors, which can be proportionally shrink down when using a smaller fabrication feature size. A mega-pixel time stamped visual motion sensor format is possible using nano-scale technology. At the same time, previous visual motion sensors normally have pixel level DC currents which prevent them to be ultra-low power. A 1 μA total bias current can lead to 3.3 W power supply for a mega-pixel array, which is high for many portable devices. The time stamped structure does not need any pixel level DC current, which makes it possible to largely optimize the power consumption.

Now referring to FIG. 8, a schematic diagram of a time stamp component 800 in accordance with one embodiment of the present invention is shown. The time stamp component 800 includes a capacitor C₁, a first switch SW₁, a second switch SW₂, a third switch SW₃, a fourth switch SW₄, a first D-flip-flop DFF₁ and a second D-flip-flop DFF₂. As illustrated in FIG. 9, first switch SW₁, second switch SW₂, third switch SW₃, and fourth switch SW₄ may comprise one or more transistors. The first switch SW₁ is connected in series between a time input (time) and the parallel connected capacitor C₁. The second switch SW₂ is connected in series between the parallel connected capacitor C₁ and the third switch SW₃. The third switch SW₃ is controlled by a read signal (read) and connected in series to a source follower SF₁, which is connected in series to an output node (out). The fourth switch SW₄ is controlled by the read signal (read) and connected in series between the output terminal \overline{Q} of the second D-flip-flop DFF₂ and an odd frame signal node (odd). The first D-flip-flop DFF₁ has a clear terminal (clear) that receives a reset signal (reset), a clock terminal (clock) connected to the edge detector (edge), a data terminal (data) connected to a voltage source (vdd), a first output terminal Q that supplies a first output signal (hold) to control the first switch SW₁ and a second output terminal \overline{Q} that supplies an inverted-first output signal (nhold) to control the second switch SW₂. The second D-flip-flop DFF₂ has a clock terminal (clock) that receives the first control signal (hold) from the first D-flip-flop DFF₁, a data terminal (data) that receives an odd-even frame signal (odd_even) and an output terminal \overline{Q} that supplies an inverted second output signal (odd_store). Note that the second D-flip-flop DFF₂ can be replaced by storing the digital value onto with a transistor gate capacitor, which further reduces the layout area.

The global time signal (time) is represented by a triangle waveform. The global time signal can be digital as well as analog, but an analog ramp signal is preferred for compact designs because it requires less layout area for the time stamp component, which is normally a capacitor. A digital memory may also be used to record the transient time in each pixel. Or, alternatively, a global clock can be used to drive a counter in each pixel to record the time. These alternatives will, however, typically require a larger layout area. In addition, the additional fast digital clock necessary to drive those digital memory components may increase the noise level of the entire sensor circuit.

The voltage across the capacitor C₁ tracks the time signal. When a moving edge is detected, the edge signal (edge) triggers the DFF₁ and the hold signal becomes high. As a result, switch SW₁ is open and holds the time voltage existing at the time the edge occurs. At the same time, nhold is low and turns on SW₂. Later on, when it is the time to read out the time stamp from this pixel, the read signal (read) becomes high and turns on SW₃ and the time_store can be read out from the pixel through source follower SF₁. At the beginning of the acquisition, the cell is reset (reset) through DFF₁ so that the internal signal hold is low and SW₂ is closed, meaning there is no time stamp recorded. At the same time, a DFF₂ is used to remember whether the recorded moving edge occurs at even frame or odd frame. The DFF₁ and DFF₂ are both edge triggered by the input signals to capture the transient point fast and accurately.

Referring now to FIG. 9, a schematic diagram of a time stamp component 900 showing more details at the transistor level in accordance with one embodiment of the present invention is shown. The time stamp component has one capacitor C₁ for time stamp voltage storage (time_store). A transmission gate formed by m1 and m2 is used to track the time signal. A shorted dummy transmission gate formed by m3 and m4, which are half sizes of that of m1 and m2 respectively, is used to reduce the charge feed through effect to improve accuracy. An output source follower containing m5, m6, and m7 provides a buffer between the capacitor C₁ and the row-column read out circuits. D-flip-flop DFF₁ is used to remember whether or not a moving edge has been detected and the resulting stored time stamp voltage has been read out. D-flip-flop DFF₂ is used to remember whether the recorded moving edge occurs at even frame or odd frame. There are five inputs for this component: reset, edge, time, odd_even, and read. There are two outputs for this component: odd and out.

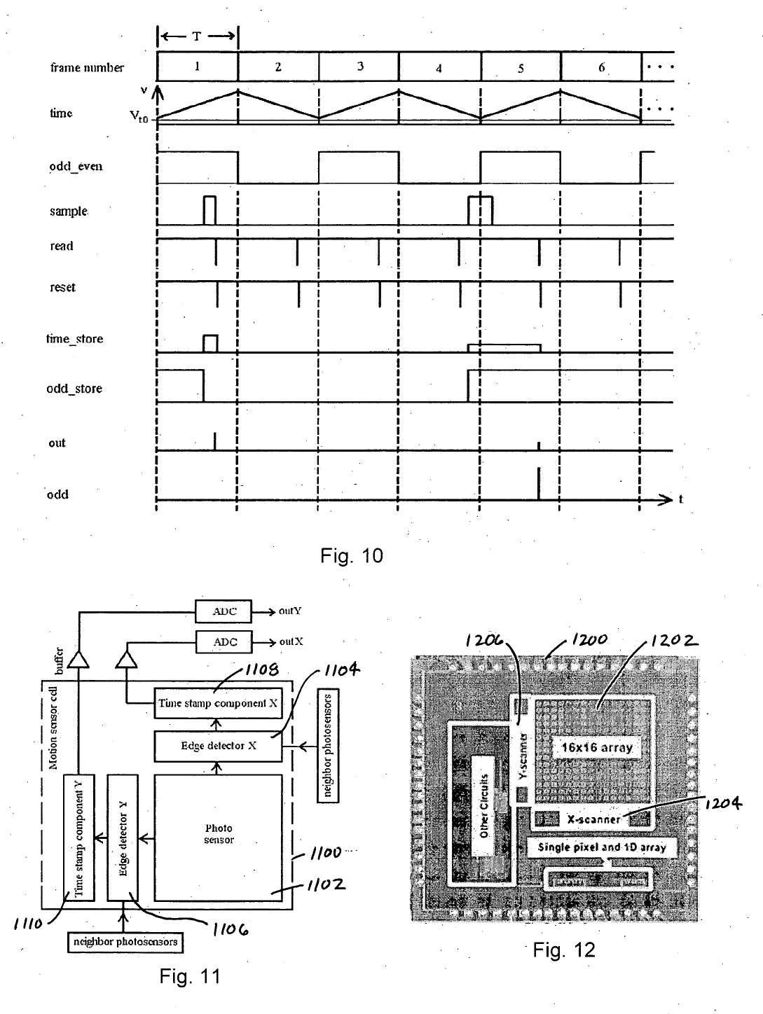

Referring now to FIG. 10, a simulated waveform of the time stamp component in accordance with one embodiment of the present invention is shown. The time signal is generated by a global ramp signal generator as a triangle wave. The period of the triangle wave should be two times of the frame sampling period T. The voltage for the time signal is Vₜᵢₘₑ=Vₜ₀+k(t−nT) for an odd frame, or Vₜᵢₘₑ=Vₜ₀+k(nT+T−t) for even frame, wherein Vₜ₀ and k are constants, n is the frame number, t is the time, and nT<t<(n+1)T. At the beginning of the acquisition, the cell is reset so that there's no time stamp recorded. The internal signal hold is low and the m1 and m2 are closed so the voltage on C₁ is tracking the input value of time. When a moving edge detected, a rising edge will be generated by the edge detector (edge), which triggers DFF₁ and sets hold to be high. The nhold changes to be low correspondingly. The transistors m1, m2 are then opened so as to hold the voltage on C₁ (time_store). Later on, when the read signal is received, the voltage stored on C₁ (time_store) will be read out from the pixel through transistor m5, m6, m7. However, if no moving edge has been recorded, even if there is a read signal, the m6 controlled by nhold will still be opened so that there is no effective output value. The change of hold signal also triggers DFF₂ to record the odd_even input at that time (odd_store). When the read signal is received, odd_store will be output as odd through m8. The DFF₁ and DFF₂ are both edge triggered by the input signals for capturing the transient point fast and accurately. The recorded time stamps can then be expressed as t=nT+(Vₜᵢₘₑₛₜₐₘₚ−Vₜ₀)/k for odd frames, or t=nT+T−(Vₜᵢₘₑₛₜₐₘₚ−Vₜ₀)/k for even frames.

For a 2-D image sensor system, the “out” signals of many pixels need to be connected together for readout. A typical method is to connect one row or one column together. However, only a single pixel is chosen at a certain time. This can be done by using an X-axis scanner and Y-axis scanner together and generate “read” signal by a simple “and” logic, i.e. “read”=X and Y. As a result, the “read” signal is only a narrow pulse for each pixel. Unlike other image sensors, which normally reset a whole column at the same time, the present invention resets each pixel right after the readout of the pixel. This way there will be only a very small portion of moving edges be missed during the short interval between the end of “read” pulse and “reset” pulse. It is easy to just use the “read” signal of one pixel as “reset” signal of its neighbor pixel for compact design.

A first example of an embodiment of a time stamp sensor pixel in accordance with the present invention will now be described. This example uses the above-described time stamp component, a prior art photosensor (see Reference No. 15 below) and a prior art edge detector (see Reference No. 16 below). Other types of photosensors and edge detectors can be used according to the specific application requirements. FIG. 11 shows the structure for the two dimensional motion sensor cell 1100 and FIG. 12 shows the layout for the motion sensor chip 1200 for this example. The time stamped sensor pixels have been fabricated in standard 0.35 μm CMOS process as a 16×16 pixel array 1202. Each pixel 1100 has one photosensor 1102, two moving edge detectors (edge detector X 1104 and edge detector Y 1106) and two time stamp components (time stamp component X 1108 and time stamp component Y 1110) for 2D motion detection, as well as input/output and power supply lines. The photosensor 1102 is a 45 μm×45 μm vertical PNP type phototransistor. Each time stamp component 1108 and 1110 occupies 40 μm×17 μm of die area. The total pixel size is 100 μm×100 μm. The chip 1200 also includes an X scanner 1204, a Y scanner 1206, various other circuits 1208 and a single pixel and one dimensional array 1210.

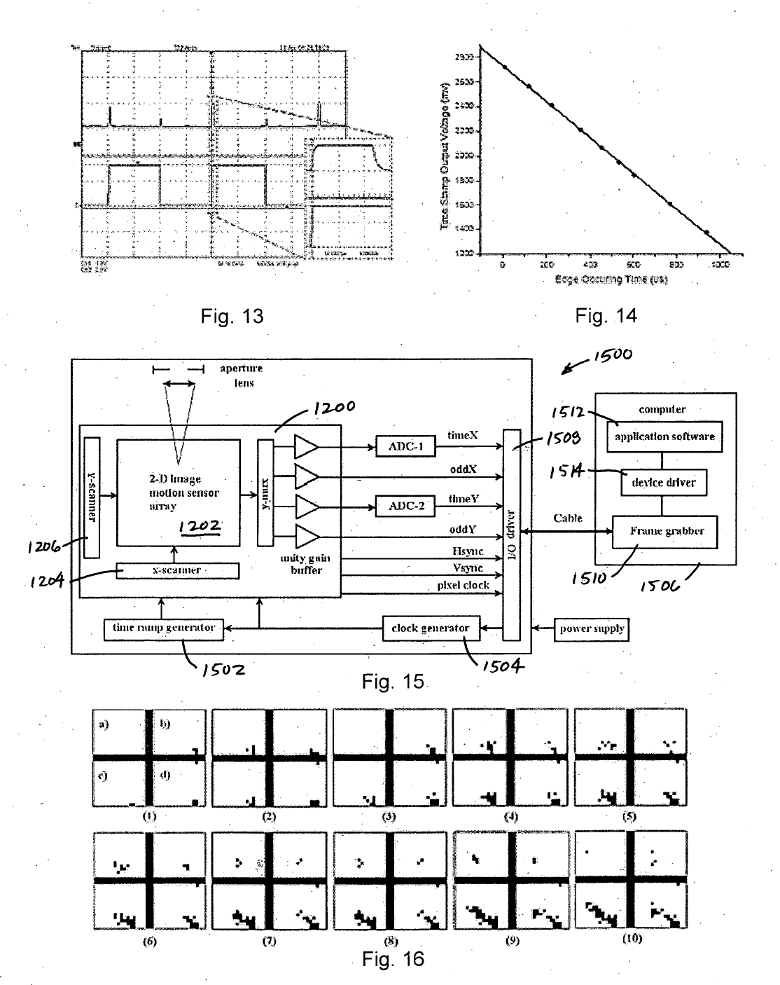

The present invention was tested by placing a rotating object in front of the chip 1200. The stimulus is a rotating fan with 18 black and white bars, which generates a faster moving edge repeating rate than the motor rotation rate. A variable quartz halogen light source was used to adjust the background luminance. The readout frame rate is set at 1000 frame per second (fps); the time resolution determined by the frame rate thus is 1 ms. The two output signals of the test pixel are measured: the recorded time stamp signal (“out”, ch1) and the recorded odd or even frame signal (“odd”, ch2) as shown in FIG. 13. For the convenience of testing, the selection pin “read” of the pixel under test is always on so that the “out” pulse is wider and its voltage level can be read more accurately after it is sent off chip. The output of the recorded timestamp values are cleared at the end of each frame so that the pixel is reset to be ready for recording upcoming edges. The “odd” signal indicates whether the moving edge appeared in the odd frame or even frame; it remains the same for the following frames until there is new edge detected. In this measurement result, it happens that the two consecutive edges appear at odd and even frames respectively. From the measured waveform it can see that there is a moving edge captured appeared around each 20 ms, which means the bar moving speed is about 50 edges per second. However, the voltage level of the time stamp signal “out” offers additional time resolution within one frame; the voltage level represents different motion edge occurring time.

To further quantify the accuracy of the time stamp, relationship between the actual edge occurring time and the recorded time stamp voltage was measured. However, the motor rotation has small vibrations, which prevents accurate determination of the actual edge occurring time. In order to accurately control the edge occurring time, a waveform generator and adjustable RC delay circuits were used to generate an adjustable impulse and to feed it directly to the test pixel as the “edge” signal. The corresponding time stamp voltage was then recorded. FIG. 14 shows the relationship between these two. It has very good linearity; further measurement shows that the residue error is only 1%. This corresponds to about 7 bits of resolution. In this measurement, the frame rate is 1000 fps, i.e. the frame period is 1 ms. A 1% error in the recorded time means that the time resolution detectable is 10 μs, which means that the timestamp structure can capture motion 100 times faster than the frame rate.

The 2-D sensor array 1202 on chip 1200 was tested using the system architecture 1500 shown in FIG. 15. Since the motion sensor chip 1200 only has a sensor array 1202 and readout structure, adequate peripheral circuits are necessary to form a complete imaging system. The output of the motion sensors are connected to two AD converters (ADC-1 and ADC-2). Global “time” signal 1502 and clock signal 1504 generators are provided externally to drive the motion sensor. The sensed data is transferred to a digital computer 1506 for imaging processing through an LVDS buffer 1508 and a frame grabber 1510. The application software 1512 interfaced with the frame grabber via a device driver 1514.

FIG. 16 shows ten frames of sampled time stamps. The 2-D motion sensor array 1202 was tested by pointing a moving lightening spot on a complex background. Each frame contains four sub-areas: a) current frame of X-direction motion sensitive time stamp, b) current frame of Y-direction motion sensitive time stamp, c) accumulated frame (16 frames combination) for X-direction motion sensitive time stamp, d) accumulated frame for Y-direction motion sensitive time stamp. When there's no motion in the scene, even if the background condition is very complex, the sensor will have no output. Once there is motion occurring, it starts to record the transient times of the moving edges. The black points in the displayed frames represents those points where edges are detected and different time stamp voltages are captured. By looking into several consecutive frames, the trace of the moving object is very clear. In addition, since the time stamp values in each pixel are different in the accumulated frame, the actual moving speed can be estimated by linear fitting. If the array is large, the shape of the moving object can be reconstructed also, as shown in FIG. 7.

A second example of an embodiment of a time stamp sensor pixel in accordance with the present invention will now be described. This example uses the above-described time stamp component, a new photosensor (described below) and a new edge detector (described below). A new compact, low mismatch spatial edge detector will now be described.

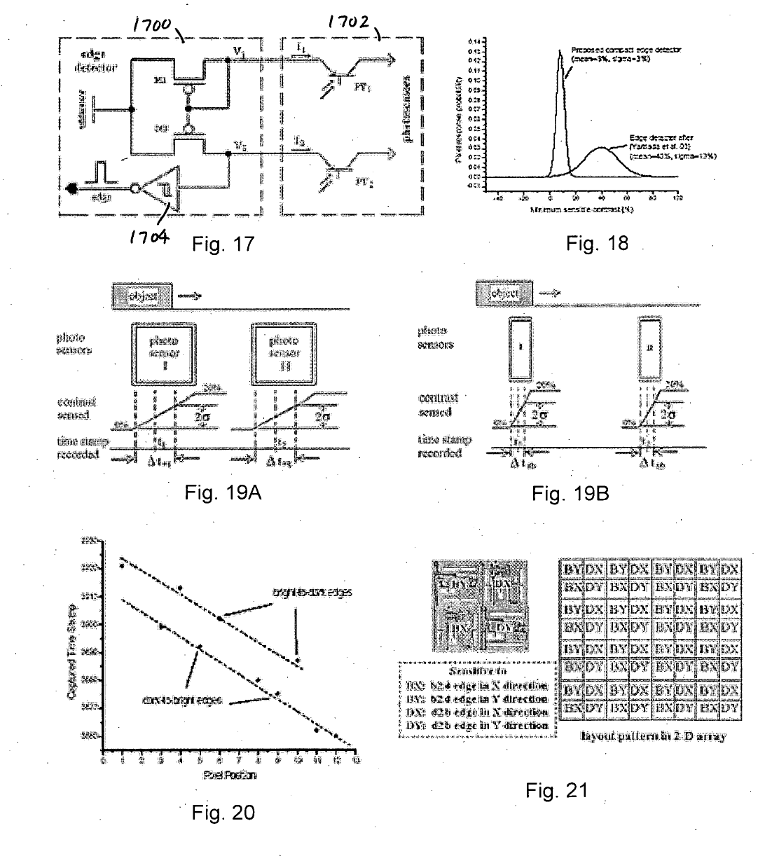

Now referring to FIG. 17, a compact spatial based edge detector 1700 in accordance with one embodiment of the present invention is shown together a schematic of the phototransistors 1702. The edge detector 1700 is a current comparing edge detector composed of a two transistor current mirror (M1 and M2) and a hysteresis inverter 1704. The edge detector 1700 is simple and compact because it only uses a two-transistor current mirror (M1 and M2). This design eliminates the two additional current mirrors used in the prior design in Reference No. 3. These two additional mirrors were used to mirror and share the output of the photosensor with neighbor pixels, so that there is only one photosensor needed in each pixel. In edge detector 1700, two phototransistors (PT₁ and PT₂) are used in each pixel so that only one current mirror necessary. The transistor count in the mirrors is reduced from 6 to 2, but the function remains the same. The simple structure allows a relatively large transistor size to be used to reduce the mismatches between two sizes of the mirror, while having smaller layout area. The performance of this small edge detector 1700 is even better than some much larger edge detectors, in terms of response speed, accuracy, noise immunity, and power consumption.

The edge detector 1700 basically compares two photocurrents (I₁ and I₂) in current mode using the current mirror. Normally when I₁≈I₂, both V₁ and V₂ will be relatively high because the photocurrent is very small, from fA to nA range. Simulation shows the output voltage V₁ and V₂ are larger than Vdd/2 through more than 120 dB of light input. As a result, the output of the hysteresis inverter 1704 is low. However, at the places where 12 is obviously larger than I₁, the output of the voltage V₂ will drop by a large amount. This triggers the hysteresis inverter 1704 output to be high. Statically, the output of the hysteresis inverter 1704 gives the spatial edges of the image when I₂>I₁. Dynamically, when there is moving objects in the scene, the positions of the spatial edges will change according to the motion. Consequently, there will be transient changes of the “edge” output of the hysteresis inverter 1704. Since the time stamp component is edge triggered, the transient time will be recorded into each time stamped pixel. The size of M1 is 10 μm×10 μm, while the size of M2 is 10.3 μm×10 μm. This additional 3% offset is used to guarantee the quiet response when I₁≈I₂, under the condition of transistor mismatches, which will be discussed below. The edge detector described here can only detect I₂>I₁ edges. Exchanging the outputs of PT₁ and PT₂ will make it detect I₁>I₂ edges. This will be used to form the separated dark-to-bright/bright-to-dark edge layout pattern, which will be discussed below.

One of the important performances of the edge detector 1700 is its contrast sensitivity. For motion detection in 2D array condition, the uniformity of the contrast is a major contributor to the accuracy of the speed measurement. Generally high contrast sensitivity is preferred, such as 5% contrast or less, but under the condition that the uniformity is acceptable. Due to the fabrication mismatches between pixels, the actual contrast sensitivity has a statistical distribution. Using normal distribution as an estimation, the distribution will have a mean value and a deviation range.

Because of the distribution caused by mismatches, the average sensible contrast cannot be biased too low. Otherwise, there will be a non-ignorable portion of pixels near or less the 0% contrast point. The points near the 0% contrast sensitivity will have noisy output even if there are no inputs. At the same time, those pixels falling into the contrast sensitivity region less than 0% may lead to malfunction. Simulation has been carried out to quantitatively analyze the effect of the fabrication mismatches over the contrast sensitivity, comparing the proposed edge detector and the edge detector in Reference No. 3. The analysis condition is 100 pA background photocurrent, which is photocurrent under bright indoor condition. Major mismatch considered here is the threshold variation. Geometric mismatches also contribute to the variation but much less than the effect of threshold, especially under carefully matching pixel layout and relatively large transistor sizes.

Referring now to FIG. 18, a graph illustrating a comparison of simulated contrast sensitivity distribution for a compact spatial based edge detector in accordance with one embodiment of the present invention and a prior art edge detector (Reference No. 3) is shown. The Yamada edge detector (Reference No. 3) has mean contrast sensitivity of 40% and standard deviation of 13%. The overall sensitivity is low, since only less than 5% of the pixels will response to 20% contrast input. Besides, the standard deviation is as large as 13%, which will cause obvious precision drop in velocity calculation. In the Yamada edge detector, the rms error of the speed measurement is 11% to 18%, which matches this analysis and verifies the contrast sensitivity non-uniformity is a major contributor to the errors in velocity measurement. The compact edge detector of the present invention shows better performance. The mean sensible contrast is 8% and the sigma is only 3%. The high sensitivity and uniformity is the result of the low-mismatches of the simple structure. The 8% mean contrast sensitivity offset is caused by the unbalanced sizes of M1 (W/L=10 μm/10 μm) and M2 (W/L=10.3 μm/10 μm), and the low threshold of the hysteresis inverter (1.1v). The same transistor sizes were used in the comparison of the Yamada edge detector and the proposed one.

Now referring to FIGS. 19A and 19B, the time stamp recording process using square and narrow par shape photosensors in accordance with one embodiment of the present invention is shown. The photosensor shape in this example of the present invention is not square, which is commonly used in most imager pixels. Instead, narrow bar shape phototransistors are used to boost the spatial resolution and make the velocity calculation more accurate. FIG. 19A illustrates the time stamp recording process using square shape photosensors. Because the photosensor spans certain area, the output current of the photosensor will change gradually even with the input of steep moving object edges. A simple estimation is that the photocurrent is proportional to the area illuminated by the incident light, thus, a linearly increasing transient photocurrent occurs. As a result, the sensed contrast between two neighbor photosensors also changes linearly. As analyzed before, the actual edge detector response has variations among the array due to the fabrication mismatches. Using the sensitivity of the spatial edge detector previously described, 2σ, or 96% edge occurring time will cover the contrast range from 2% to 14%. This variation causes corresponding variation in recorded time stamp values, equivalent to time variation of Δt_{sq}. In FIG. 19B, narrow-bar shape photosensors are used instead of the square ones. If the sensor is n time narrower than the square ones in horizontal direction, the transient speed of the sensed contrast is n times faster than that of the square photosensor pixels. Since the distribution of the contrast sensitivity keeps the same, the recorded time stamp values will be compressed n times in time domain. This leads to a 2σ variation of Δt_{nb} n times smaller than Δt_{sq}. When doing velocity calculation using linear fit, the recorded time stamp values will be located in the range n times closer to the ideal curve than that using square photosensors.

Referring now to FIG. 20, a double edge problem observed in previous motion sensor chips is shown (i.e., the mixed response of the dark-to-bright (d2b) edges and bright-to-dark (b2d) edges). Especially when there is relatively small object moving fast in the scene, the d2b and b2d edges can occur at very small time interval. On the other side, the contrast sensitivity of the edge detector is not uniformed, which makes some edge detectors more sensitive to d2b edges while others more sensitive to b2d edges. The result is mixed d2b and b2d edges. Using a small bright point as the stimuli, if the motion speed is high, the groups of time stamp points in b2d edge is close to those from d2b edge, thus making it difficult for the program to choose right points to calculate speed using multi-point linear fit.

Now referring to FIG. 21, a layout pattern of a spatial edge based time stamped motion sensor in accordance with one embodiment of the present invention is shown. The detection of d2b edges with the b2d edges is separated. At the same time, the edge detectors have one type sensitive to x-axis motion and another sensitive to y-axis motion. As a result, there are four types of pixels in- one group. Since the pattern of the pixel group layout is known, only those pixels with the same type are compared to calculate speed, thus, there is no mixed b2d/d2b edge problems. The velocity is then calculated based on linear fitting of four consecutive even points or odd points. In some cases when the input contrast is low or motion speed is very high, there are only three available consecutive points but velocity calculation can still be successfully done if the three points show near linear motion.

As a result, the present invention also provides a visual motion sensor array that includes four or more visual motion cells. Each visual motion cell includes a photosensor, an edge detector connected to the photosensor and a time stamp component connected to the edge detector. The visual motion cells can be arranged into an array of pixel groups. Each pixel group includes a first pixel that is sensitive to a bright-to-dark edge in a X direction (BX), a second pixel that is sensitive to the bright-to-dark edge in a Y direction (BY), a third pixel that is sensitive to a dark-to-bright edge in the X direction (DX) and a fourth pixel that is sensitive to the dark-to-bright edge in the Y direction (DY).

Referring now to FIG. 22, a chip block diagram and readout structure in accordance with one embodiment of the present invention is shown. The pixel readout structure is similar to those used in large scale 2D CMOS digital image sensors. Different from typical digital imagers with rolling shutter, which reset pixels row by row, this design connects the “read” signal of one pixel to the “reset” pin of its neighbor so that the pixel is ready to receive new input immediately after the sampled data being read out. Assuming a 1 Meg readout pixel clock at 100 fps, each pixel is only occupied in 2 clock periods for readout/reset and spends larger than 99.98% time for edge detection. I/O buffers have been included, as well as the logic circuits to synchronize the pixel clock with the analog “time” signal.

Accordingly, the present invention provides a visual motion sensor chip 2200 that includes an array of visual motion cells 2202, an X-axis 2204 and Y-axis 2206 scanner, a multiplexer 2208, a synchronization signal generation logic and output buffer 2210, and an input buffer and synchronization logic 2212. Each visual motion cell 2214 includes a photosensor 2216, an edge detector 2218 connected to the photosensor 2216, and a time stamp component 2220 connected to the edge detector 2218 and provides an output signal. The X-axis scanner 2204 is connected to the array of visual motion cells 2202. The Y-axis scanner 2206 connected to the array of visual motion cells 2202. The multiplexer 2208 is connected to the array of visual motion cells 2202 and provides a time output, an image output and an odd frame output. The synchronization signal generation logic and output buffer 2210 provides a vertical synchronization signal, a horizontal synchronization signal and a pixel clock signal, and is connected to the X-axis scanner 2204 and the Y-axis scanner 2206. The input buffer and synchronization logic 2212 receives an odd-even frame signal, a time signal and a clock signal, and is connected to the X-axis scanner 2204, the array of visual motion cells 2202 and the multiplexer 2208. The visual motion sensor chip 2200 can be integrated into a device used for video compression, robotics, vehicle motion control or high speed motion analysis.

For the velocity measurement, a high-speed moving object with controlled speed is necessary. The visual motion sensor chip 2200 was tested using a laser pointer pointing to a rotating mirror, which is mounted on a smooth running motor. The laser is reflected by the mirror to a target plane that is one meter away; the bright dot on the target plane is the object. The advantage of this test setup is that the torque of the motor is minimal so that it runs very smoothly even at such high speed as 3000 RPM. Also, the bright moving point is like the target in particle image velocity (PIV) system, which is a possible application for the proposed sensor.

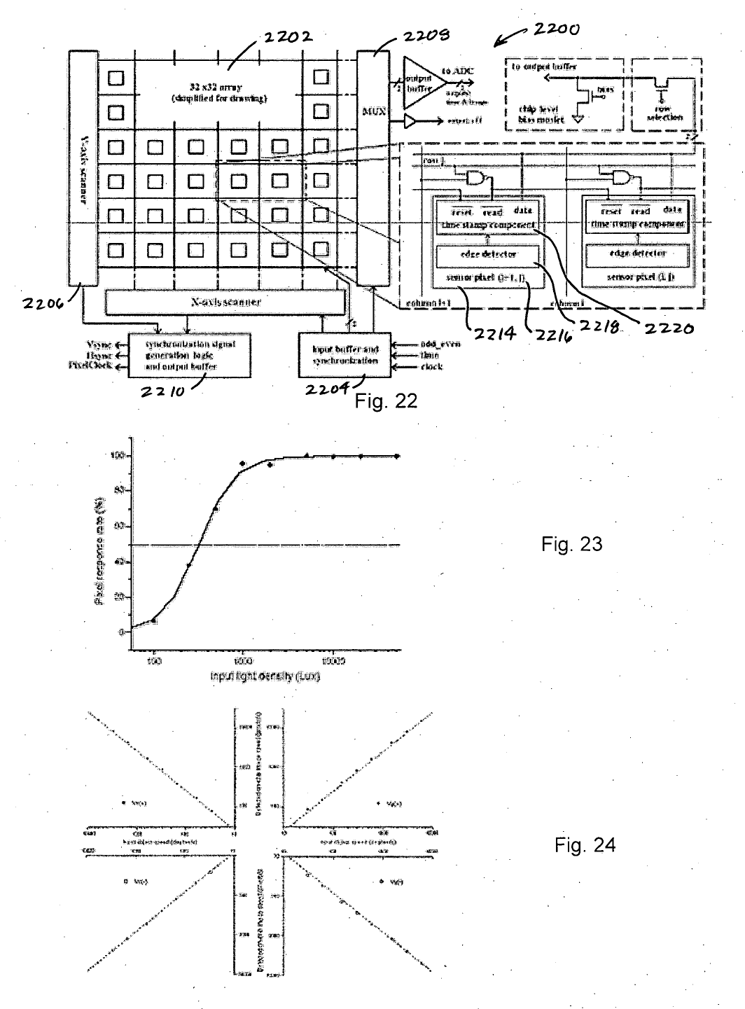

Now referring to FIG. 23, a measured pixel response rate of the spatial edge detector in 2D array (with 50% contrast input) in accordance with one embodiment of the present invention is shown. As shown, the edge detector functions from 400 to 50000 Lux (under the condition of larger than 50% pixel response rate @ 50% input contrast, with lens F-number 1.4). Further testing using slow and fast moving object as stimuli shows that it responds to angle speeds as low as 1 degree/s and as high as 6000 degrees/s, with focal length f=10 mm. At the same time, it is quiet when there is no motion, even with complex background and flashing fluorescent light.

Referring now to FIG. 24, a measured velocity in horizontal and vertical directions in accordance with one embodiment of the present invention is shown. As shown, the present invention achieves less than 5% rms variation for middle speed range (300 to 3000degree/s), and less than 10% rms variation for low and high speed range. Compared with digital camera plus computer systems, one of the key advantages of the time stamped motion sensor is that it can capture motion in much higher time resolution than the frame rate.

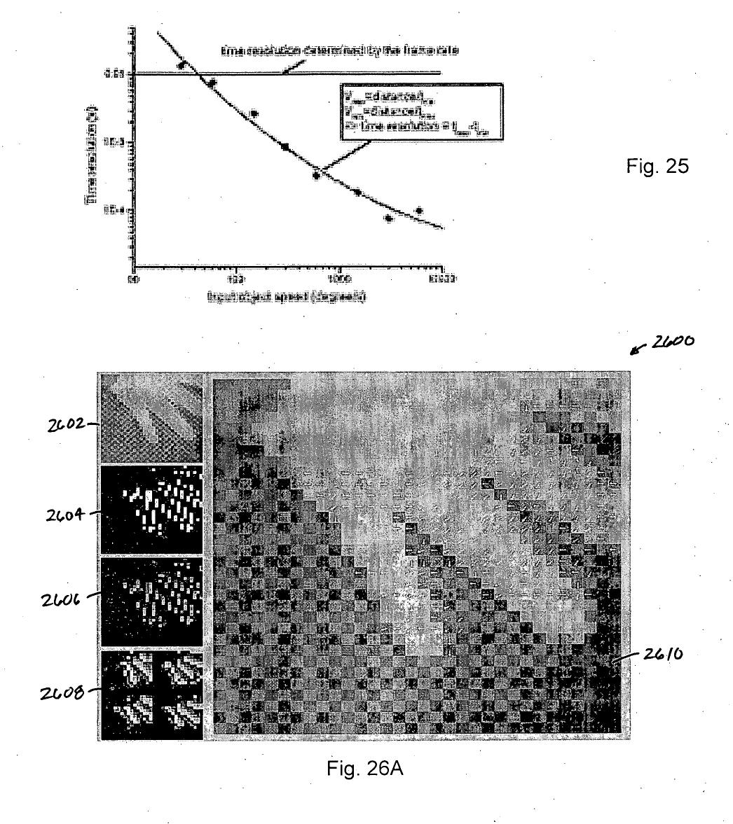

Now referring to FIG. 25, an equivalent time resolution based on measured velocity accuracy in accordance with one embodiment of the present invention is shown. A digital camera running at 100 fps can only have time resolution of 10 ms, while the time stamped motion sensor of the present invention obtained about 0.1 ms time resolution running at the same frame rate.

Referring now to FIG. 26A, a measured 2-D optical flow 2600 of a moving hand in accordance with one embodiment of the present invention is shown. The first small picture 2602 is the original video frame captured by the log scale imager integrated together with the motion sensor. The sensor runs at 100 fps and the second small picture 2604 shows the detected moving parts at current frame, which basically occur at the edge of hand. The third picture 2606 shows the time stamp values of each detected moving point by different grey levels. The last small picture 2608 is a combined version of four time stamp frames with separated view area for BX, DX, BY, DY edges, which shows more clearly the trace of the motion, with brighter points for more recently occurring edge points. Finally, the large picture 2610 presents the motion vectors calculated based on the four frame combination of time stamps. From the four optical flow pictures, the moving directions of the hand and fingers are clear.

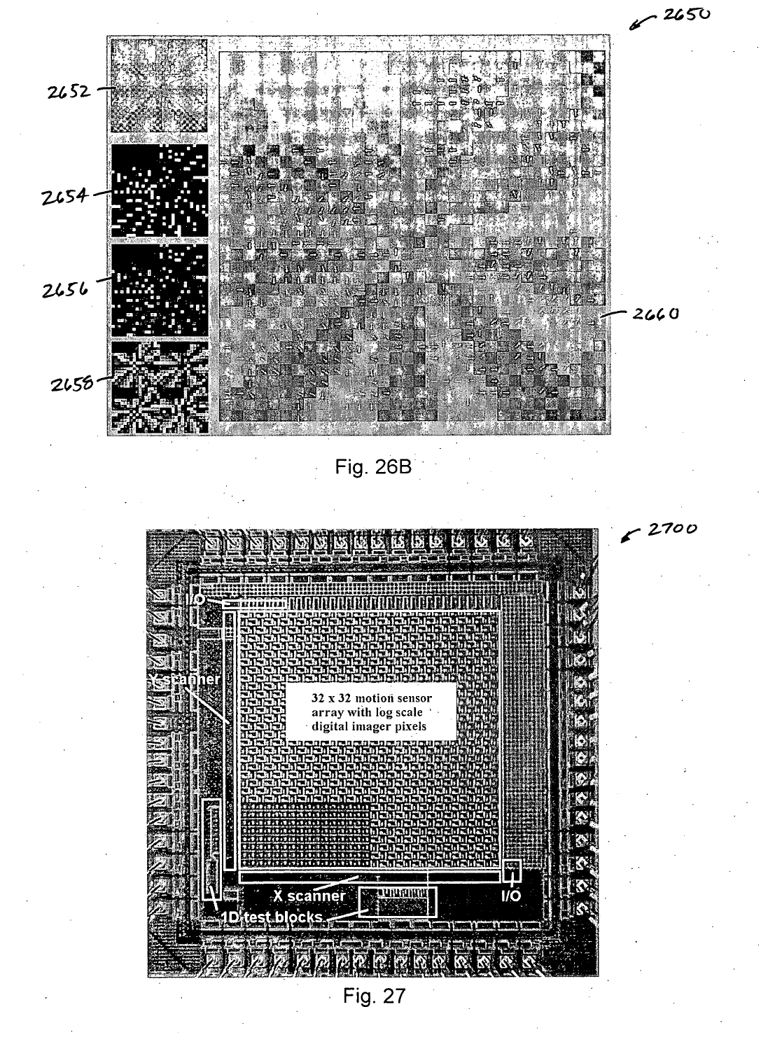

Another example of the measured 2-D optical flow 2650 is shown in FIG. 26B. It is a fast running fan with repeatable pattern of rotation movement. However, since the array resolution of the test chip is low, the vectors calculated from local points may not always reflect the correct motion vector of the whole object. The first small picture 2652 is the original video frame captured by the log scale imager integrated together with the motion sensor. The second small picture 2654 shows the detected moving parts at current frame, which basically occur at the edge of fan blades. The third picture 2656 shows the time stamp values of each detected moving point by different grey levels. The last small picture 2658 is a combined version of four time stamp frames with separated view area for BX, DX, BY, DY edges, which shows more clearly the trace of the motion, with brighter points for more recently occurring edge points. Finally, the large picture 2660 presents the motion vectors calculated based on the four frame combination of time stamps. From the four optical flow pictures, the moving directions of the fan blades are shown. The correct motion of the whole object can be determined using additional algorithms. The photo of the chip 2700 of the embodiment of the present invention described above is shown in FIG. 27.

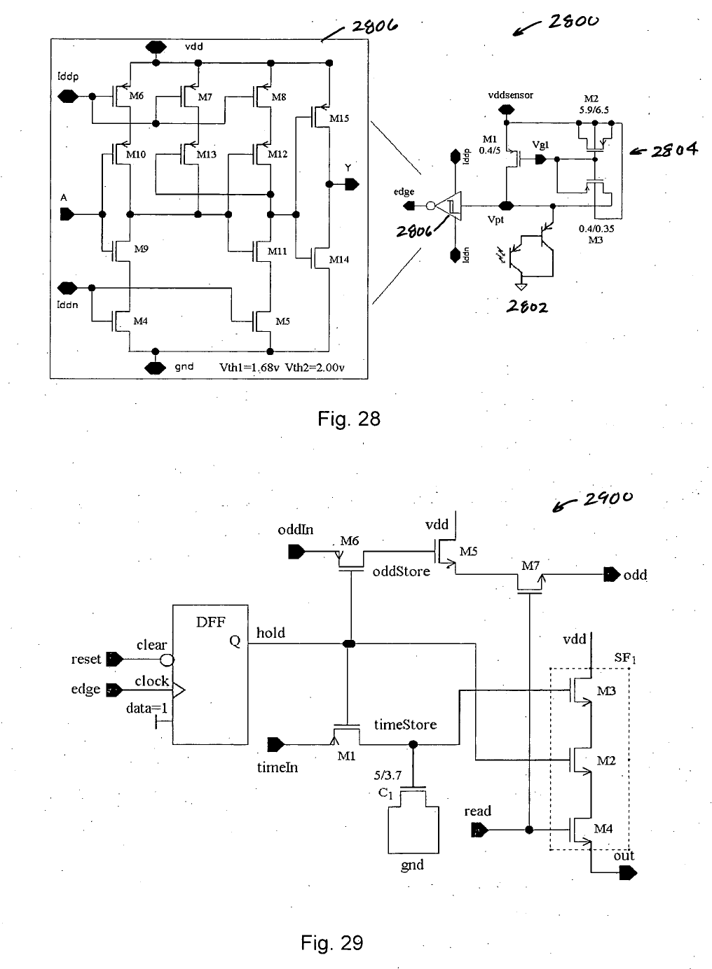

Another embodiment of the present invention will now be discussed, which further takes advantage of the time stamped structure to achieve ultra-low power consumption. FIG. 28 shows the design of a nano-power edge detector 2800 in accordance with the present invention. It contains a phototransistor 2802, a 3-transistors photocurrent sensing stage 2804 (M1, M2 and M3), and a hysteresis inverter 2806 as digitizing stage. The photocurrent flows through transistor M1, while M2 acts as a capacitor and M3 as a large resistor. When the photocurrent is constant, the voltage the gate of M1, Vg1, equals to the voltage at the drain of M1, Vpt; thus, M1 is diode connected. Since the photocurrent is normally very small, M1 works in weak inversion region. As a result, the equilibrium voltage of Vpt changes in log scale with the photocurrent. It can remain higher than 2V when the photocurrent varies within certain 120 dB range. When motion occurs, the light density at the image edges increases suddenly, causing Vpt to drop. However, because of the large capacitor at node Vg1, the current going through M3 is not large enough to charge the gate voltage, Vg1 quickly. As a result, there is a difference between the voltages at Vg1 and Vpt. Since Vg1 charges slowly, it acts as a ‘memory’ to remember a delayed signal. Therefore, the |V_{DS}| of M1 increases to balance the increased photocurrent. Meanwhile, the W/L ratio of M1 is small and it requires a large increase of |V_{DS}|. This causes the voltage Vpt to drop significantly so as to trigger the hysteresis inverter to output a rising edge.

A hysteresis inverter 2806 is used to digitize the edge signal. A current-clamping hysteresis inverter is designed, as shown in FIG. 28. A current source and a current sink are added into each column of a traditional hysteresis inverter. As a result, the maximum transient current will be limited by the bias current. Simulation shows that the peak current goes up to hundreds of μA with meta-status analog input in a traditional hysteresis inverter, while the current-clamping hysteresis inverter limits the current within nano-ampere range. The selection of the clamping bias current is a balance between the slew rate, the motion-static power and the transient power. Simulation shows that 1 μs rising time can be achieved using 10 nA clamping current, with the condition of 1 nA background photocurrent and 1 ms rising edge input with 50% contrast. This is fast enough for most consumer applications. However, for high-speed motion analysis applications, higher clamping bias current may be necessary.

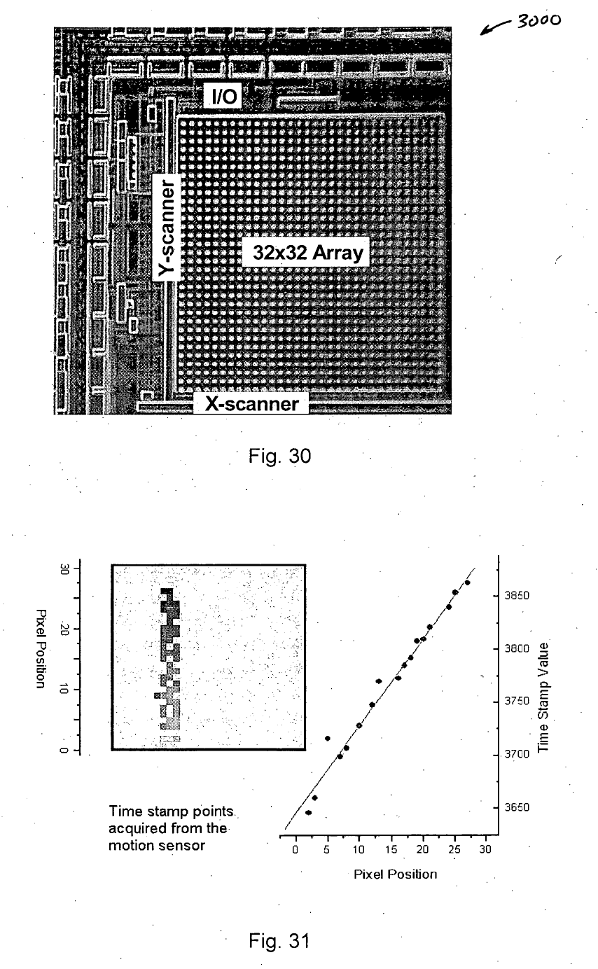

Now referring to FIG. 29, the circuit implementation of a nano-power time stamp component 2900 in accordance with present invention is shown. The global ‘time’ signal is represented by a triangle waveform. The voltage across the capacitor C1 tracks the ‘time’ signal. When there is a moving edge detected, the ‘edge’ signal triggers the DFF and the ‘hold’ signal becomes high. As a result, switch M1 is open and C₁holds the ‘time’ voltage at which the ‘edge’ occurs. At the same time, M2 turns on. Later on, when it is the time to read out the time stamp from this pixel, the ‘read’ signal becomes high and turns on M4 and the ‘timeStore’ can be read out through the source follower SF1. At the beginning of the acquisition, the cell is reset through DFF so that the internal signal ‘hold’ is low and M2 is closed, meaning there is no time stamp recorded. Meanwhile, the gate capacitor of M5 is used to remember whether the recorded moving edge occurs at even frame or odd frame. This structure intrinsically does not need any DC bias current, while in previous design in FIG. 9, a simplified DFF was used which costs 384 nW where there is no edge appearing and 308 nW where there is 1 edge appearing per frame. A static DFF is used in this paper and the power drops to 0.46 nW when there is no motion and 2.0 nW when there is 1 edge appearing at each frame. A 32×32 test chip has been fabricated in standard CMOS 0.35 μm process. A photo of the chip 3000 is shown in FIG. 30.

Referring now to FIG. 31, a frame of the readout data from the 2D sensor is shown when a bright spot traveling quickly from bottom to top. It shows that the pixels around column 10 detects motion; the grey levels in each pixel represents the time when there is an edge passing that pixel (time stamp value). The time stamp values are re-plotted at the right for pixels at column 10. Since the time stamp values are the motion edge occurring time, the moving speed then can be estimated using a linear fit of these points. The chip is measured to dissipate 3 μA to 11 μA total averaged current, depending on the frequency of the motion. Within 0 to 50000 Lux illumination range, the luminance has almost ignorable effect on the power consumption, as the photocurrent is very small (less than 1 nA/pixel) and there is no in-pixel photocurrent amplification path. The averaged pixel power consumption is only 10 nW to 35 nW.

The performance of the present invention is superior to prior art motion sensors, such as Reference No. 3, 9 and 10. The 32×32 visual motion sensor demo chip based on the present invention can have a pixel size of 70 μm×70 μm in a standard 0.35 μm CMOS process. Such a device can measure up to 6000 degree/s with a focal length f=10 mm and has less than 5% rms variation for middle range velocity measurement (300 to 3000 degree/s) and less than 10% rms variation for high velocity (3000 to 6000 degree/s) and low velocity (1 to 300 degree/s) measurement. The device has a power consumption of less than 40 μW/pixel using a single power supply. In the ultra-low power embodiment of the present invention described above, the pixel power consumption was further lowered down to 35 nW/pixel, which is hundreds of times lower than that of other structures. Besides, this structure is good for scaling down with new fabrication processes to implement large scale 2D arrays with low power consumption. Other characteristics of the device include a fill factor greater than or equal to 32%, a frame readout rate greater than or equal to 100 fps, a peak time resolution less than or equal to 77 μs at 100 fps with 3000 degrees/s input, and a dynamic range for luminance of 400 to 50000 Lux at larger than 50% pixel response rate at 50% input contrast with a lens F-number 1.4.

Some of the many possible applications for the present invention will now be discussed.

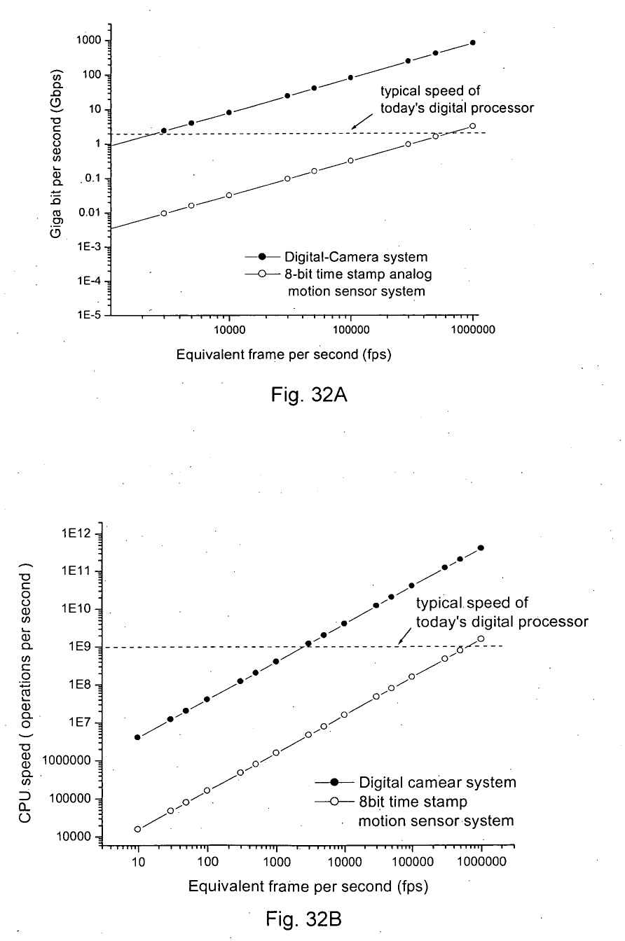

High speed motion analysis - The basic function of high speed motion analysis is to obtain the optical flow field from the sampled video sequences. It is very useful in modern aerodynamics and hydrodynamics research, combustion research, vehicle impact tests, airbag deployment tests, aircraft design studies, high impacts safety component tests, moving object tracking and intercepting, etc. The traditional solution in the state-of-the-art machine vision industry uses digital camera plus digital computer system for high speed motion analysis. It needs to transfer the video data frame by frame to digital processor and do motion analysis algorithms based on it. There are two major bottle necks: data transfer load and computational load. FIG. 32A and 32B give an example of how the time stamped motion sensor can dramatically lower down these two loads. In FIG. 32A, minimum data transfer load for digital camera system is calculated as W×H×F×B/10⁹ (Gbps); minimum data transfer load for time stamp motion detection system with 8-bit timestamp component is calculated as W×H×F×B/10⁹/256 (Gbps), wherein W is frame width, H is frame height, F is FPS (frame per second), B is the bit depth of the pixel color. In FIG. 32B, to calculate the computional load for digital camera system, at least 4 neighbor pixels needs to be compared to find out basic motion information. So, the minimum computational load is W×H×F×4 unit operation/second. For time stamp motion detection system with 8-bit time stamp component, frame rate reduced to 1/256 for detecting the same speed. So, the minimum computational load can be estimated as W×H×F×4/256 unit operation/second. Noticing that the curves are shown in log scale, the timestamp motion sensor can lower the two major loads 100 times or more. The timestamp motion sensor also has the special feature of catching fast motion by slow frame rate. It has the potential of continuously measure high resolution motion in microsecond time-resolution, which is far beyond existing commercial products.

Real-time MPEG video compression—Another possible application for the time stamped motion sensor is to aid the real-time MPEG video compression. One of the most computational intensive tasks of the MPEG4 video compression is motion estimation. The standard FS (full search) algorithm may cost as high as 80% total computational power of the video encoding system. This is not acceptable, especially in portable devices. The timestamp motion sensor can be very helpful in the real-time motion estimation.

The basic algorithm for the MPEG motion estimation is to search the best matching macroblocks within a specified displacement area. The computational load for FS algorithm can be calculated as (2p+1)²N², wherein p is the maximum displacement of the moving picture block, while N² is the size of a macroblock. A typical configuration is p=N=16. When a video frame with the size of W×H is used for motion estimation by dividing it to N² macroblocks, the total load can be calculated as
Load (FS)=(2p+1)²N²×(W/N)×(H/N)=(2p+1)²×W×H
For a MPEG4 CIF format W=352, H=288, using p=16, then,
Load (FS)=(2×16+1)²×352×288=1.1×10⁸ (unit operation per frame)
The unit operation here normally means an absolute of subtracting and a summarizing operation. For a standard frame rate FPS=30, the total real-time motion estimation load is
Load (FS)=3.3×10⁹ (unit operation per second)

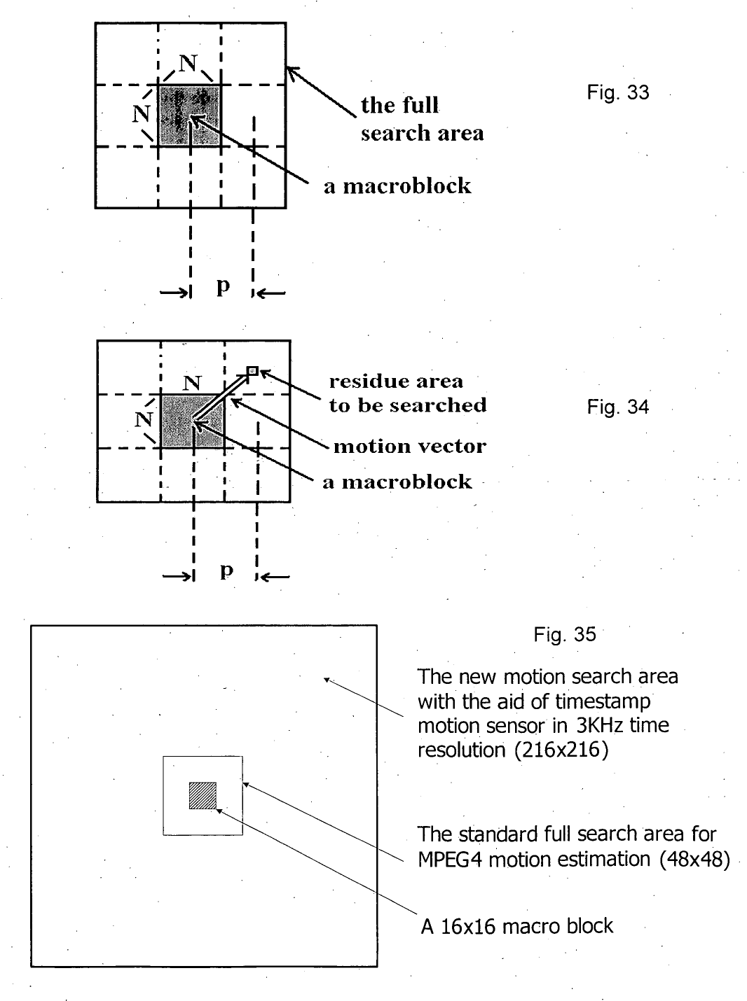

When a time stamped motion sensor is used, a motion vector can be measured for each pixel. Based on the averaged motion vectors from all the pixels in one macroblock, a nominal vector for this macroblock can be estimated. Assuming the measured motion vectors are accurate, the nominal vector will be very near the position of the best matching block. Considering that there might be residue offset errors exist, the FS algorithm can be applied within a small area near the nominal vector position. Assuming that p=N=16, W=352, H=288, and the nominal vector has 25% accuracy (which is a generous condition and easy to achieve by the timestamp motion sensor), only (p/4)²=4×4 area near the nominal vector indicated position needs to be searched. FIG. 33 gives the searching area using FS algorithm and FIG. 34 illustrates the searching area using time stamped motion sensor. The total computational load can then be calculated as,

$$\begin{matrix}Load ( timestamp motion estimation ) = ( W \text{/} N ) \times ( H \text{/} N ) \times {N}^{2} \times {( p \text{/} 4 )}^{2} + \\ motion vector calculation overhead \\ = W \times H \times {( p \text{/} 4 )}^{2} + \\ motion vector calculation overhead \\ = 352 \times 288 \times 4 \times 4 + \\ motion vector calculation overhead \\ = 1.62 \times {10}^{6} + motion vector \\ calculation overhead \\ ( unit operation per frame )\end{matrix}$$

While the motion vector calculation overhead can be estimated as

$$\begin{matrix}Load ( overhead ) = motion vector calculation for all pixels + \\ motion vector averaging for each macroblocks \\ = 2 \times W \times H + 2 \times ( W \text{/} N ) \times ( H \text{/} N ) \times {N}^{2} \\ = 4 \times W \times H = 4 \times 352 \times 288 \\ = 0.405 \times {10}^{6} ( unit operation per frame )\end{matrix}$$

So the total load is

$$\begin{matrix}Load ( timestamp motion estimation ) = 2.03 \times {10}^{6} \\ ( unit operation per frame ) \\ = 6.09 \times {10}^{7} \\ ( unit operation per second \\ @ 30 fps )\end{matrix}$$

Compared with the full search algorithm, the timestamp motion estimation computational load is
3.3×10⁹/(6.09×10⁷)=54 times lower.
A simplified formula can be written as

$$\begin{matrix}Load ( FS ) / load ( timestamp ) = [ {( 2 p + 1 )}^{2} \times W \times H ] / [ {k}_{1} \times W \times H \times \\ {( {k}_{2} \times p )}^{2} ] \\ = {( 2 p + 1 )}^{2} / ( {k}_{1} \times {k}_{2}^{2} \times {p}^{2} )\end{matrix}$$

Wherein, k₁=(overhead ratio) which is 1.25 in the above calculation, k₂=(motion vector accuracy) which is 25% in the above calculation.

In addition, since the timestamp motion sensor can achieve better than 25% motion. vector accuracy, it is quite possible that a good matching point has been found after several initial tries at the central of the residue area. In that case, further searching is no necessary so that the actual ratio of the computational load saving is even larger.

Furthermore, because the dynamic range of the speed measurement based on time stamp architecture is wide, there is actual no limit of the displacement. In previous the FS algorithm, it is quite possible an object image may jump out of the (2p+1)² range between the reference frame and the estimated frame. When that happens, the FS algorithm cannot find a good matching, which results in discontinued low quality video quality and/or lower compress ratio. On the contrary, the timestamp motion vector can easily catch the fast jump and leads to more clear motion pictures. The searching area with the aid of the timestamp motion sensor is actually even larger than with multi-frame time stamp combination technique. In other words, the motion sensor of the present invention does not only increase the processing speed, lowers the power consumption, but also improves the video quality. FIG. 35 illustrates an example of enlarged search area using time stamped visual motion sensor.

Several fast algorithms for MPEG motion estimation have been reported to largely reduce the power consumption of the motion estimation task to less than 5 percent of the FS algorithm. However, most of them have the following drawbacks: (1) the fast speed and low power consumption are obtained by trading off the video quality; (2) the motion searching area is still limited as that of the standard FS algorithm so they are not good for fast action movie recording; (3) most of these methods usually only good for low resolution videos such as MPEG4 simple profile (355×288). They are not effective for high resolution video, such as DVD (720×480) and HDTV (1920×1080) standards, because the computation load for motion estimation is not proportional to the image size but much larger. For example, for 1920×1080 HDTV @30 fps, the load for standard full search algorithm is

$$\begin{matrix}Load ( FS , HDTV ) = 256 \times 128 \times 1920 \times 1080 \times 30 \\ = 2038 GOPS \\ = 617 \times Load ( FS , CIF )\end{matrix}$$

Wherein GOPS means giga operations per second.

When a time stamped motion sensor with 10% nominal motion vector accuracy are used to aid the motion estimation, only about an 8×8 residue area needs to be searched for best matching. The new load will be

$$\begin{matrix}Load ( TimeStamp , HDTV ) = residue motion estimation + \\ motion vector calculation overhead \\ = 8 \times 8 \times 1920 \times 1080 \times 30 + \\ 4 \times 1920 \times 1080 \times 30 \\ = 4.23 GOPS \\ = 0.0021 \times Load ( FS , HDTV )\end{matrix}$$

It is possible that other optimization algorithms can be applied, such as the GDS (Gradient Descent Search), on the 8×8 residue area so that the final load can be even lower. A conventional HDTV motion estimation processor using FS algorithm costs more than 1200 mW even with 1/4 sub-sampling technique. Using time stamped motion sensor together with optimized algorithm, such as GDS, the present invention may have less than 50mW with equal or better quality than that of 1/1 sampling FS algorithm.

Real-time optically feedback motion control—Visual information is very useful for most living creatures to control their movement. It is also very important in the artificial world. As a result, the present invention can be useful for the intelligent motion control of the robots, vehicles and aircrafts.

### REFERENCES

1. Y. W. Huang, S. Y. Ma, C. F. Shen, and L. G. Chen, “Predictive Line Search: An Efficient Motion Estimation Algorithm for MPEG-4 Encoding Systems on Multimedia Processors”, IEEE Trans. on circuit and systems for video technology, vol. 13, No. 1, pp. 111-117, January 2003.

2. C. Mead, Analog VLSI and Neural Systems, Reading, Mass.: Addison-Wesley, 1989.

3. Keiichi Yamada and Mineki Soga, “A Compact Integrated Visual Motion Sensor for ITS Applications”, IEEE Trans. On Intelligent Transportation systems, Vol. 4, No. 1, pp.35-42, 2003.

4. R. Etienne-Cummings, “Biologically Inspired Visual Motion Detection in VLSI”, International Journal of Computer Vision, 44(3), pp.175-198, 2001.

5. Moini, Vision Chips, Kluwer Academic Publishers, Boston/Dordrecht/London, ISBN 0-7923-8664-7, 2000.

6. R. R. Harrison and C. Koch, “A Robust Analog VLSI Motion Sensor Based-on the Visual System of the Fly”, Autonomous Robots 7(3): pp. 211-224, November 1999.

7. R. Etienne-Cummings, J. Van der Spiegel, P. Mueller, and M. Z. Zhang, “A foveated silicon retina for two dimensional tracking,” IEEE Trans. on Circuits and Systems II: Analog and Digital Signal Processing, vol. 47, pp. 504-517, June 2000.

8. G. L. Barrows, K. T. Miller, and B. Krantz, “Fusing neuromorphic motion detector outputs for robust optic flow measurement”, in Proceedings of Intl. Joint Conf. on Neural Networks, pp. 2296-2301, 1999.

9. G. Indiveri, J. Kramer, and C. Koch, “System implementations of analog VLSI velocity sensors”, Micro, IEEE, vol. 16, pp. 40-49, October 1996.

10. R. Etienne-Cummings, J. Van der Spiegel, and P. Mueller, “A focal plane visual motion measurement sensor”, IEEE Trans. on Circuits and Systems I: Fundamental Theory and Applications, vol. 44, pp.55-66, January 1997.

11. J. Kramer, G. Indiveri, and C. Koch, “Analog VLSI motion projects at Caltech”, Advanced Focal Plane Arrays and Electronic Cameras, Proc. SPIE 2950, pp. 50-63, 1996.

12. M. Arias-Estrada, D. Poussart, and M. Tremblay, “Motion vision sensor architecture with asynchronous selfsignaling pixels”, Fourth IEEE Intl. Workshop on Computer Architecture for Machine Perception, pp.75-83, October 1997.

13. G. Indiveri, P. Oswald, J. Kramer, “An adaptive visual tracking sensor with a hysteretic winner-take-all network”, IEEE Intl. Symp. on Circuits and Systems, vol.2, pp.32⁴-327, May 2002.

14. A. Moini, “Neuromorphic VLSI systems for visual information processing: drawbacks”, Knowledge-Based Intelligent Information Engineering Systems, Third Intl. Conf., pp. 369-372, 1999.

15. R. W. Sandage and J. A. Connelly, “Producing phototransistors in a standard digital CMOS technology”, Circuits and Systems, ISCAS, Connecting the World IEEE Intl. Symp., vol. 1, pp.369-372, 2000.

16. G. B. Zhang and J. Liu, “A robust edge detector for motion detection”, IEEE Intl. Symp. on Circuits and Systems, pp. 45-48, May 2002.

17. U.S. Pat. No. 5,781,648.

18. U.S. Pat. No. 5,998,780.

19. U.S. Pat. No. 6,023,521.

Although preferred embodiments of the present invention have been described in detail, it will be understood by those skilled in the art that various modifications can be made therein without departing from the spirit and scope of the invention as set forth in the appended claims.

What is claimed is:

1. A visual motion sensor cell comprising:
a photosensor;
an edge detector connected to the photosensor; and
a time stamp component connected to the edge detector.

2. The visual motion sensor cell as recited in claim 1, wherein the edge detector receives inputs from the photosensor and generates a pulse when a moving edge is detected.

3. The visual motion sensor cell as recited in claim 2, wherein the time stamp component tracks a time signal and samples a time voltage when the moving edge is detected.

4. The visual motion sensor cell as recited in claim 3, wherein the sampled time voltage is stored until the sampled time voltage is read.

5. The visual motion sensor cell as recited in claim 1, wherein the edge detector is further connected to one or more neighboring photosensors.

6. The visual motion sensor cell as recited in claim 1, wherein the time stamp component comprises:
a first switch connected in series between a time input and a parallel connected capacitor;
a second switch connected in series between the parallel connected capacitor and a third switch;
the third switch controlled by a read signal and connected in series to a source follower, which is connected in series to an output node;
a first D-flip-flop having a clear terminal that receives a reset signal, a clock terminal connected to the edge detector, a data terminal connected to a voltage source, a first output terminal that supplies a first output signal to control the first switch and a second output terminal that supplies an inverted first output signal to control the second switch;
a second D-flip-flop having a clock terminal that receives the first control signal from the first D-flip-flop, a data terminal that receives an odd-even frame signal and an output terminal that supplies an inverted second output signal; and
the fourth switch controlled by the read signal and connected in series between the output terminal of the second D-flip-flop and an odd frame signal node.

7. The visual motion sensor cell as recited in claim 6, wherein the first, second, third and fourth switches each comprise one or more transistors.

8. The visual motion sensor cell as recited in claim 6, wherein the second D-flip-flop is replaced by a transistor having a gate connected capacitor to supply the inverted second output signal.

9. The visual motion sensor cell as recited in claim 1, wherein the photosensor comprises a narrow bar shaped photosensor.

10. The visual motion sensor as recited in claim 1, wherein the edge detector detects an edge when a current differential exists between the two phototransistors or photodiodes.

11. The visual motion sensor cell as recited in claim 1, wherein the edge detector comprises a two transistor mirror circuit connected to the photosensor and a hysteresis inverter, which is connected in series to the time stamped component.

12. The visual motion sensor cell as recited in claim 11, wherein the two transistor mirror circuit comprises two transistors having a size difference.

13. The visual motion sensor cell as recited in claim 12, wherein the size difference is greater than or equal to 3%.

14. The visual motion sensor cell as recited in claim 11, wherein the two transistor mirror circuit is connected to one or more neighboring photosensors.

15. The visual motion sensor cell as recited in claim 1, wherein the visual motion sensor comprises a pixel.

16. The visual motion sensor cell as recited in claim 15, wherein the pixel is sensitive to a bright-to-dark edge in a X direction, the bright-to-dark edge in a Y direction, a dark-to-bright edge in the X direction or the dark-to-bright edge in the Y direction.

17. The visual motion sensor cell as recited in claim 15, wherein the pixel has a size less than or equal to 70 μm by 70 μm or a power consumption less than or equal to 40 μW.

18. A visual motion sensor array comprising four or more visual motion cells, each visual motion cell comprising a photosensor, an edge detector connected to the photosensor and a time stamp component connected to the edge detector.

19. The visual motion sensor array as recited in claim 18, wherein the visual motion cells are arranged into an array of pixel groups, each pixel group comprising a first pixel that is sensitive to a bright-to-dark edge in a X direction, a second pixel that is sensitive to the bright-to-dark edge in a Y direction, a third pixel that is sensitive to a dark-to-bright edge in the X direction and a fourth pixel that is sensitive to the dark-to-bright edge in the Y direction.

20. A visual motion sensor chip comprising:
an array of visual motion cells, each visual motion cell comprising a photosensor, an edge detector connected to the photosensor, and a time stamp component connected to the edge detector and provides an output signal;
a X-axis scanner connected to the array of visual motion cells;
a Y-axis scanner connected to the array of visual motion cells;
a multiplexer connected to the array of visual motion cells and that provides a time output, an image output and an odd frame output;
a synchronization signal generation logic and output buffer that provides a vertical synchronization signal, a horizontal synchronization signal and a pixel clock signal, and is connected to the X-axis scanner and the Y-axis scanner; and
an input buffer and synchronization logic that receives an odd-even frame signal, a time signal and a clock signal, and is connected to the X-axis scanner, the array of visual motion cells and the multiplexer.

21. The visual motion sensor chip as recited in claim 20, wherein the chip is integrated into a device used for video compression, robotics, vehicle motion control or high speed motion analysis.

22. The visual motion sensor chip as recited in claim 20, wherein the chip has one or more of the following characteristics:
a single power supply less than or equal to 3.3 volts;
a power consumption less than or equal to 40 μW;
a pixel size less than or equal to 70 μm by 70 μm;
a fill factor greater than or equal to 32%;
a frame readout rate greater than or equal to 100 fps;
a dynamic range for speed from 1 degree/s to 6000 degrees/s;
a velocity measurement accuracy of less than 5% rms variation for 300 to 3000 degrees/s and less than 10% rms variation for 1 to 300 degrees/s and 3000 to 6000 degrees/s;
a peak time resolution less than or equal to 77 μs at 100 fps with 3000 degrees/s input; or
a dynamic range for luminance of 400 to 50000 Lux at larger than 50% pixel response rate at 50% input contrast with a lens F-number 1.4.

23. A method of detecting visible motion comprising the steps of:
receiving an image signal from a photosensor;
tracking a time signal;
determining whether a moving edge is detected in the image signal; and
sampling a time voltage from the time signal when the moving edge is detected.

24. The method as recited in claim 23, further comprising the steps of:
storing the sampled time voltage; and
outputting the sampled time voltage when a read signal is received.

25. The method as recited in claim 23, wherein the time signal comprises a triangle waveform.

26. The method as recited in claim 23, further comprising the step of estimating a motion of a visible object by comparing the sampled time voltages from an array of photosensors.

27. The method as recited in claim 23, wherein the photosensor is sensitive to a bright-to-dark edge in a X direction, the bright-to-dark edge in a Y direction, a dark-to-bright edge in the X direction or the dark-to-bright edge in the Y direction.
